(19) United States
(12) Patent Application Publication
(10) Pub. No.: US 2026/0215975 A1
(43) Pub. Date: Jul. 30, 2026

(54) ABSORBENT ARTICLE

(71) Applicant: Essity Hygiene and Health Aktiebolag, Göteborg (SE)

(72) Inventors: Philip BLOMSTRÖM, Göteborg (SE); Alice FLODIN VELTMAN, Göteborg (SE); Anna KNÖS, Göteborg (SE)

(73) Assignee: Essity Hygiene and Health Aktiebolag, Göteborg (SE)

(21) Appl. No.: 19/147,536

(22) PCT Filed: Mar. 16, 2023
(86) PCT No.: PCT/EP2023/056770

§ 371 Date: Jul. 11, 2025

(51) Int. Cl. A61F 13/496; A61F 13/15; A61F 13/511

(52) U.S. Cl. CPC A61F 13/496; A61F 13/15268; A61F 13/51121; A61F 2013/15276

(57) ABSTRACT

A washable and reusable undergarment including one or more fabric panels forming a front region, a back region and an intermediate region extending between the front and back regions. The undergarment includes an absorbent assembly including a wearer facing top layer of a textile material, a moisture barrier, and an intermediate layer(s) being superimposed along a height axis, perpendicular to the longitudinal axis and the transversal crotch axis, between the top layer and the moisture barrier. The absorbent assembly is permanently attached to at least one out of the one or more of fabric panels. The top layer and/or the one or more fabric panels are of natural material and the top layer has length in the longitudinal direction and an extension beyond the longitudinal length of the barrier and intermediate layer(s) of the absorbent assembly.

### TECHNICAL FIELD

The disclosure relates to a washable and reusable undergarment comprising one or more fabric panels and an absorbent assembly permanently attached to at least one of the fabric panels. The absorbent assembly comprises at least a wearer facing top layer of a textile material, a moisture barrier, and at least an intermediate layer.

### BACKGROUND

An absorbent article is worn for the purpose of absorbing body fluids such as urine and vaginal fluids. There is disposable as well as washable and reusable absorbent articles. A disposable absorbent article is disposed after use, and a washable and reusable absorbent article comprises textile fabrics of woven or knitted materials and after use it is washed or laundered to be restored before reuse. A washable and reusable absorbent undergarment conventionally comprises an integral and permanently attached absorbent assembly to be located in the crotch region of the wearer when the garment is worn. Reusable absorbent undergarments have to date mainly been of synthetic material and for low and moderate volumes of body fluids. For sustainability reasons there has been an increased demand from users for reusable absorbent undergarments and there is also a demand for such articles comprising natural materials suitable for heavier flows of body fluids, increasing the demand for technical solutions enabling the construction of such garments.

### SUMMARY

The present disclosure is based on the insight that there is a need for a solution for a washable and reusable absorbent undergarment comprising natural materials enabling a secure, and yet aesthetically pleasing, attachment of the absorbent assembly to a fabric panel of the undergarment.

The present invention provides a solution by a providing a washable and reusable undergarment according to claim 1.

As such, there is provided a washable and reusable undergarment comprising one or more fabric panels forming a front region, a back region and an intermediate region extending between the front and back regions, the front region and the back region being joined such that the undergarment forms a waist opening and a pair of leg openings. A central longitudinal axis (y) of the undergarment is defined along the one or more fabric panels of the undergarment from the back region and towards the front region, and a transversal crotch axis (x) of the undergarment is defined in a direction extending between the leg openings and so as to divide the intermediate region into a front intermediate region extending longitudinally between the transversal crotch axis (x) and a front end of each leg opening, and a rear intermediate region extending longitudinally between the transversal crotch axis (x) and a rear end of each leg opening, the transversal crotch axis (x) being perpendicular to the central longitudinal axis (y). The undergarment further comprises an absorbent assembly comprising a wearer facing top layer of a textile material, a moisture barrier, and at least one intermediate layer being superimposed along a height axis (z), perpendicular to the longitudinal axis (y) and the transversal crotch axis (x), between the top layer and the moisture barrier. The absorbent assembly is permanently attached to at least one out of the one or more fabric panels in at least part of the intermediate region by a set of one or more bonding members. The top layer and/or the one or more fabric panels are of natural material and the top layer has length in the longitudinal direction and an extension (F) beyond the longitudinal length of the barrier and the intermediate layer(s) of the absorbent assembly.

The top layer and/or the one or more fabric panels are of natural textile material. Natural materials are selected from the group consisting of cotton, wool, silk, cellulose, regenerated cellulose, rayon, viscose, modal, lyocell, Tencel, bamboo, hemp, flax, ramie, coir or banana. Textile materials of natural materials are normally thicker and bulkier than textile materials of synthetic fibers. In addition, the natural materials have shorter fibers, and it is therefore more difficult to attach a textile material of natural fibers to another textile material being of natural or synthetic fibers, if one would like to avoid attaching the layers together by stitching.

The top layer has length in the longitudinal direction and an extension (F) beyond the longitudinal length of the barrier and the intermediate layer of the absorbent assembly. The extension of the top layer may be at least 5 mm longer than the barrier and the intermediate layer(s) of the absorbent assembly, such as at least 10 mm longer than the barrier and the intermediate layer(s) of the absorbent assembly and may be less than 100 mm longer than the barrier and the intermediate layer(s) of the absorbent assembly, such as less than 30 mm longer than the barrier and the intermediate layer(s) of the absorbent assembly. The extension may thus be within the range 5-100 mm, such as 10-30 mm. The mentioned range of the extension ensures a reliable permanent attachment of the top layer to a fabric panel when one of the layers, or both the top layer and a fabric panel, are of natural materials.

The extension of the top layer may be both in the front and the rear part of the top layer. The extension may be along substantially the full width of the top layer. The extension of the top layer may be within the front and rear intermediate regions of the undergarment only.

The top layer may be attached to a fabric panel in the extending part of the top layer by one or more bonding members selected from a fastening tape, an area of adhesive or formed at least partially via a thermocompression process.

The fastening tape may be an elastic bonding tape, sealing tape, glue tape or an elastic film. The bonding member may overlap a border between an outer edge region of the extension of the top layer and a fabric panel, providing a nice and smooth reliable and permanent attachment of the top layer to a fabric panel.

The bonding member may be located between the top layer and a fabric panel, at and near an edge region of the extending top layer, providing a reliable and permanent, yet aesthetically pleasing, attachment of the top layer to a fabric panel without stitching the layers.

Adhesive may similarly be located between the top layer and a fabric panel, at and near an edge region of the extending top layer. Adhesive may be a spray-on adhesive, a liquid curable adhesive, and/or a thermoset adhesive. The thermocompression process may be by applying heat and/or pressure to the adhesive material during the manufacture of the undergarment.

The adhesive may comprise one or more bonding lines. One or more bonding lines may be arranged to extend along at least part of the front and rear edge of the extending top layer, such as to extend along essentially the entire front and rear edge of the extending top layer.

One or more bonding lines may be arranged to extend along at least part of the side edges of the absorbent assembly, such as to extend along the side edges of the absorbent assembly in the intermediate region of the undergarment. The one or more bonding line may be arranged to extend along essentially the entire side edges of the absorbent assembly.

The one or more bonding lines may be continuous bonding lines. Optionally, the one or more bonding lines may be intermittent bonding lines.

A set of bonding members is arranged to attach the absorbent assembly to the at least one out of the one or more of fabric panels and may be arranged at least along part of the side edges of the absorbent assembly. For example, the set of bonding members may be arranged to attach the absorbent assembly to the at least one out of the one or more of fabric panels along essentially the entire side edges of the absorbent assembly.

The side edges of the absorbent assembly may be arranged at or near the respective leg opening of the undergarment in at least the front intermediate region.

Thus, for example, in some variants the set of bonding members may be arranged to attach the absorbent assembly to the at least one out of the one or more fabric panels along the side edges, the front edge, and the rear edge of the absorbent assembly.

The shape of the absorbent assembly may be adapted for example to the design of the undergarment, and in view of the need for sufficient absorption and satisfactory fit.

In some variants, the side edges may comprise concave portions, as seen towards the central longitudinal axis y. For example, the side edges may be generally concave, thus allowing for the shape of the absorbent assembly being relatively wider towards the front and rear of the undergarment, and narrower at a crotch portion of the undergarment comprising the transversal crotch axis, as mentioned in the above.

In some variants, the front edge may be convex from the transversal crotch axis. As such, at the location of the central longitudinal axis y, the front edge may protrude towards the front of the undergarment beyond the side edges of the absorbent assembly. This may be beneficial for the fit of the undergarment to the body and contribute to the avoidance of unwanted creases in the fabric panels.

The rear edge of the absorbent assembly may be shaped to allow for suitable fit and comfort to a user.

The intermediate region of the undergarment may comprise at least one out of the one or more fabric panels, the fabric panel having an exterior side facing away from the wearer, and an interior side facing in a direction towards the wearer. The absorbent assembly may be located on the interior side of the at least one fabric panel.

As outlined in the above, the absorbent assembly comprises a wearer facing textile top layer, a moisture barrier, and at least one intermediate layer being superimposed along a height axis, perpendicular to the central longitudinal axis and the transversal crotch axis, between the top layer and the moisture barrier.

The intermediate layer may be at least an absorbent layer. The intermediate layer may be at least a wicking layer and an absorbent layer, such as two or more absorbent layers.

As such, the absorbent assembly may comprise at least two layers plus the moisture barrier, which in some variants is formed by a moisture barrier layer. Such an absorbent assembly may be particularly suited for a moderate flow of body fluids.

The absorbent assembly may comprise a wearer facing top layer, a moisture barrier, and at least two intermediate layers. As such, the absorbent assembly comprises at least three layers plus the moisture barrier, which in some variants is formed by a moisture barrier layer. Accordingly, the capacity of the absorbent assembly to absorb relatively high flows of body fluid may be further improved, for example to provide an absorbent assembly suited for a heavy flow of body fluids.

For example, the absorbent assembly may comprise no more than nine layers. As such, the absorbent assembly may comprise two, three, four, five, six, seven or eight intermediate layers. One of the intermediate layers may be a wicking layer.

The longitudinal and transversal extension (i.e. the outer shape) of the absorbent assembly per se is defined by the longitudinal and transversal extension of the wearer facing top layer and the moisture barrier.

All the intermediate layers and the barrier of the absorbent assembly have a shorter longitudinal extension than the top layer.

The top layer may be a weft knit. The top layer may be of a Jacquard technique. The top layer may have a basis weight of 80-250 gsm. The top layer may be of natural material or synthetic material or blends thereof.

The basis weight of the absorbent layer(s) may for example be in the range 150-500 gsm. The absorbent layer(s) may be of natural or synthetic material or blends thereof, such as cotton, polyester, polyamide, or a mixture thereof. The absorbent layer may be of a hydrophilic French terry material.

The intermediate layer may be a wicking layer. The wicking layer may be arranged immediately adjacent the top layer. The basis weight of the wicking layer may be 180-250 gsm. For example, the wicking layer may be of polyester, polyamide, elastane or a mixture thereof. For example, the wicking layer may be a jersey knit.

### BRIEF DESCRIPTION OF THE DRAWINGS

The disclosure will be described in greater detail below with reference to the figures shown in the appended drawings, wherein

FIG. 1 shows a front view of a variant of an absorbent undergarment comprising an absorbent assembly;

FIG. 2 shows a top view from the wearing facing side of the undergarment of FIG. 1 when in a flat laid-out state with the joints between the front rear region and the front region being removed; and

FIG. 3 is a schematic cross-sectional view of a portion of the absorbent undergarment of FIG. 2 through the absorbent assembly.

FIG. 4 is a schematic cross-sectional view of an alternative absorbent undergarment.

### DETAILED DESCRIPTION

Different aspects of the present disclosure will be described more fully hereinafter with reference to the enclosed drawings. The embodiments of the absorbent undergarment disclosed herein can, however be realized in many different forms, such as different sizes and absorption levels, and should not be construed as being limited to the aspects set forth herein. In all figures in the following detailed description, the same reference numerals will be used to indicate the same elements.

As used herein, the term “absorbent undergarment” refers to garments that are worn and intended to be placed against the skin of the wearer to absorb and contain body fluids such as urine and vaginal fluids including menstrual fluid. The absorbent undergarment is an underwear of a fabric or textile for female use. The undergarment according to the present disclosure is intended to be laundered or otherwise restored after use for reuse as a sanitary article.

By “washing” it is meant that the absorbent undergarment may be cleaned by laundering. The absorbent undergarment may thus be subjected to an aqueous solution containing detergent without losing its structural features. This aqueous solution may be heated as part of the laundering process, e.g. to 30° C. or 60° C. The undergarment may be washed and reused at least 10 times.

The terms “fabric” and “textile” as used in the present disclosure may refer to single or multiple layers of fabrics. The fabric may be knitted or woven.

Knitting is a method of constructing a fabric by interlocking a series of loops of one or more yarns. There are two major classes of knitting namely warp and weft knitting.

Weaving is a method or process of interlacing two yarns of similar materials so that they cross each other at right angles to produce woven fabric. The warp yarns run lengthwise in the fabric and the filling threads (weft) or picks, run from side to side.

Jacquard is a system of weaving or knitting that utilizes a versatile pattern mechanism to produce intricate designs by using punch cards controlling the patterns produced. Jacquard knit may be valid for circular, flatbed, warp or weft knit.

By “permanently attached”, it is meant that the absorbent assembly is not intended to be separated from the undergarment before, during, or after use.

The top layer may be constructed of any suitable textile fabric, including naturally derived fibers selected from the group consisting of cotton, wool, silk, cellulose, regenerated cellulose, rayon, viscose, modal, lyocell, tencel, bamboo, hemp, flax, ramie, coir or banana. Alternatively, the top layer may be constructed of synthetic fibers selected from the group consisting of polyamide, acrylic, polyester or elastane, such as a mixture of polyester and elastane. Further, the top layer may be constructed of a blend or a mixture of naturally derived and/or synthetic fibers. The fibers may be recycled fibers. The materials used for construction of the top layer should be soft and non-irritating to the skin and be readily penetrated by any body fluids. According to the present disclosure, the top layer should be launderable. The top layer may comprise between 20% and 100% naturally derived fibers, more preferably between 40% and 100% naturally derived fibers and most preferably between 60% and 100% naturally derived fibers. The top skin-facing layer comprises a water-permeable material thus allowing the body fluids to migrate to the underlying absorbent layer or layers. The top layer may have a basis weight of 80-250 gsm.

A wicking layer may be provided underneath the top layer. The wicking layer has wicking features to allow the moisture to spread away from the wearer and into the absorbent layer or layers. Further, wicking enables an efficient spread of the moisture therefore allowing the moisture to be received in a wider area of the underlying one or more absorbent layers. This feature is particularly relevant for users suffering from stress incontinence as spurts of urine may cause the absorbent layer to be saturated rapidly at the point of impact. By spreading the volume of the fluid over a wider receiving area in the absorbent layer, the wicking features of the wicking layer increase the overall absorptive capacity of the absorbent layer. The wicking layer may be constructed of any suitable fabric, including naturally derived fibers or mixtures thereof selected from the group consisting of cotton, wool, silk, cellulose, regenerated cellulose, rayon, viscose, modal, lyocell, Tencel, bamboo, hemp, flax, ramie, coir or banana. Alternatively, the wicking layer may be constructed of synthetic fibers or mixtures thereof selected from the group consisting of polyamide, acrylic, polyester or elastane. Further, the wicking layer may be constructed of a blend or a mixture of naturally derived and/or synthetic fibers. The fibers may be recycled fibers. According to the present disclosure, the wicking layer should be launderable. The wicking layer may comprise between 20% and 100% naturally derived fibers, more preferably between 40% and 100% naturally derived fibers and most preferably between 60% and 100% naturally derived fibers. The basis weight of the wicking layer may be 180-250 gsm.

The absorbent assembly for use in an absorbent undergarment may contain one or multiple absorbent layers, capable of absorbing liquid and releasing the liquid during laundering. Release of the absorbed liquid may be beneficial as it enables the absorbent undergarment to be restored for reuse. Reusable absorbent undergarments provide a more sustainable alternative to the commonly used disposable hygiene articles that are not intended to be re-used. The absorbent layer may comprise any material capable of absorbing fluid, such as woven or nonwoven microfiber or polymer knits, fabric formed from hydrophilic fibers, absorbent fibers or powders. The absorbent layer may be of natural, synthetic or recycled fibers as described above for the other ingoing layers but may be of cotton, polyester and polyamide and contain odor treatment.

The absorbent layer may also comprise a material having an open cell porous structures such as high loft or synthetic fibers having reservoir properties. The absorbent layer may comprise between 20% and 100% naturally derived fibers, more preferably between 40% and 100% naturally derived fibers and most preferably between 60% and 100% naturally derived fibers. The basis weight of each absorbent layer may be 150-500 gsm. There may be one or more, such as two, absorbent layers in the absorbent assembly.

The liquid-impermeable barrier layer may comprise any suitable material or combinations of material that prevents liquid from migrating from the absorbent assembly to the fabric layer. The liquid-impermeable barrier layer may comprise a hydrophobic woven or nonwoven material having inherent hydrophobic properties, or that has been treated to become hydrophobic. Examples of hydrophobic materials for treating the barrier layer are polymers such as silicone, polyurethane and combinations thereof. The liquid-impermeable barrier layer may comprise a microporous polymer film, e.g. a polyethylene, PTFE or polyurethane film, or combinations thereof. Laminates of polymer films and nonwoven materials may also be used. The liquid-impermeable barrier layer may be a coating of a moisture-impermeable material. The coating may be a polymer such as urethane wax on the surface facing away from the wearer. The liquid-impermeable barrier layer may be of polyurethane.

The liquid-impermeable barrier layer is preferably breathable, to allow vapor to escape from the absorbent undergarment, while preventing liquid from passing through the fabric layer. Further, the liquid-impermeable barrier layer may be constructed of an elastic material, thus providing the absorbent assembly with the required flexibility to adapt to the user's anatomy and movements. This improved fit increases the wearer's comfort during use and helps to prevent leakage from migrating through the undergarment's leg openings.

The fabric of the one or more fabric panels may be constructed of any suitable fabric, including naturally derived fibers and mixtures thereof selected from the group consisting of cotton, wool, silk, cellulose, regenerated cellulose, rayon, viscose, modal, lyocell, tencel, bamboo, hemp, flax, ramie, coir or banana. Alternatively, the fabric may be constructed of synthetic fibers or mixtures thereof selected from the group consisting of polyamide, acrylic, polyester. Further, the fabric may be constructed of a blend or a mixture of naturally derived and/or synthetic fibers. The fibers may be recycled fibers. Herein, recycled fibers originate from reclaimed pre- or post-consumer waste materials that are converted into new fibers through a textile recycling process to create upcycled new textiles. The fabric may comprise a stretchable fabric, e.g. elastane, so that the absorbent undergarment can provide a firm fit while at the same time adapting to the wearer's movements thus preventing any leakage from migrating through the leg openings and keeping the absorbent assembly in place. The fabric is preferably breathable to allow vapor to escape from the wearer's skin and from the absorbent structure.

The layers of the absorbent assembly and the undergarment body fabric may be bonded by conventional stitching, gluing techniques or via a bonding film such as tape.

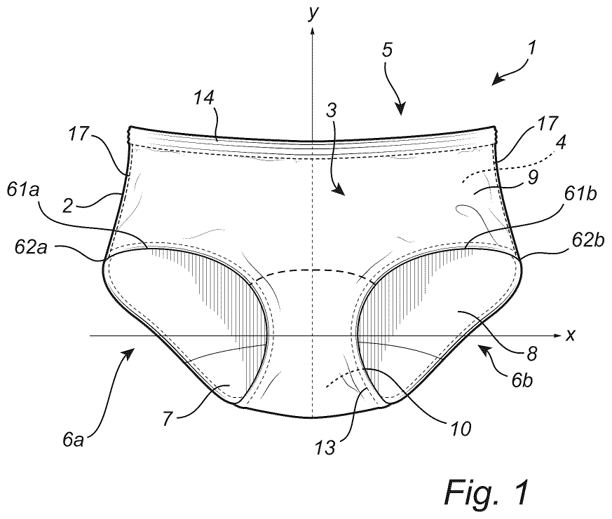

FIG. 1 illustrates as an example of a variant of a washable and reusable absorbent undergarment 1 in the form of a panty. As outlined in the above, the undergarment 1 as proposed herein may however have various designs, such as briefs, boxer, hipster, high waist, string, Brazilian, or other suitable undergarment designs. The undergarment 1 is designed primarily for female use.

The undergarment 1 may comprise one or more fabric panels 2 forming a front region 3, a back region 4 and an intermediate region 7 extending between the front and back regions 3, 4. The front region 3 and the back region 4 are joined such that the undergarment 1 forms a waist opening 5 and a pair of leg openings 6a, 6b. As such, the front region 3 will be visible from the front of the user when the undergarment 1 is worn, and the back region 4 will be visible from the back of the user when the undergarment 1 is worn.

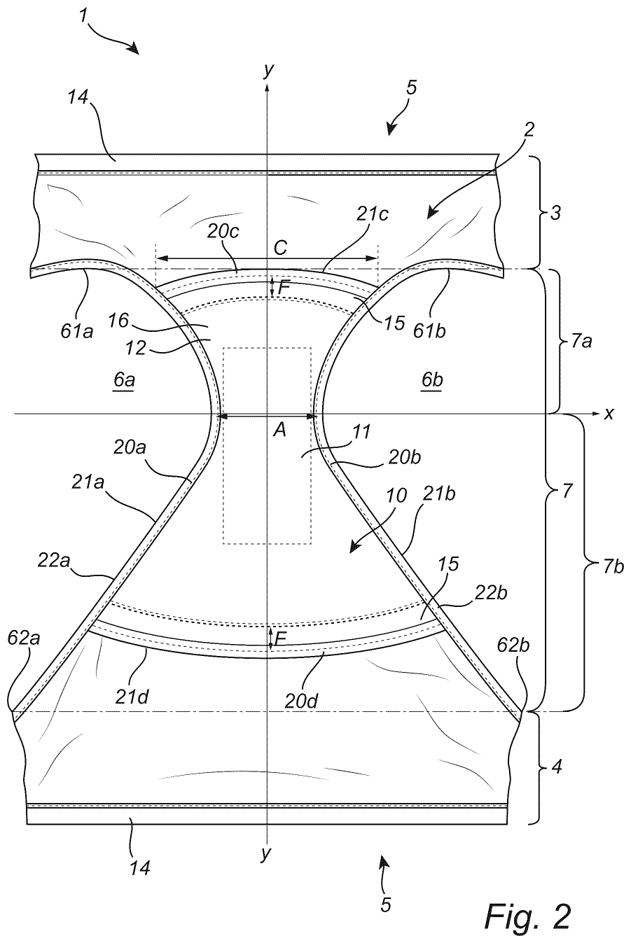

The undergarment of the FIG. 1 and FIG. 2 example comprises one fabric panel 2, which is cut to form the front region 3, the back region 4 and the intermediate region 7. However, in other variants, a plurality of fabric panels may be joined to form the front region 3, the back region 4 and the intermediate region 7. For example, the undergarment 1 may comprise a front panel, a back panel and an intermediate panel. The one or more fabric panels may all comprise the same fabric, giving the undergarment a unitary appearance, or the one or more fabric may comprise different fabrics, for example to provide aesthetically pleasing undergarments comprising e.g. lace fabric.

To join the front region 3 and the back region 4 to form the waist opening 5 and the leg openings 6a, 6b, a pair of side joints 17 may be provided, as in the illustrated example. The side joints 17 and any other joints joining the one or more fabric panels 2 may be conventional joints in the art, such as seams made by conventional adhesive and/or mechanical bonds such as conventional adhesive or stitching techniques.

A central longitudinal axis y of the undergarment 1 is defined along the one or more fabric panels 2 of the undergarment 1 from the back region 4 and towards the front region 3. As such, when an undergarment 1 is seen from the front side as in the example of FIG. 1, the central longitudinal axis y would be directed towards the waist opening 5 of the undergarment 1. However, when the undergarment 1 is seen from the back side (not shown), the central longitudinal axis y would be directed away from the waist opening 5 of the undergarment 1.

In FIG. 2, the absorbent undergarment 1 of FIG. 1 is shown in a flat laid-out state, where the side joints 17 of the undergarment 1 are removed. As seen in this laid-out state, the central longitudinal axis y is defined along the one or more fabric panels 2 and in a direction from the rear 4 of the undergarment 1 towards the front 3 of the undergarment 1. In FIG. 2, the absorbent undergarment 1 is shown from a wearer-facing side of the article 1. As illustrated in FIG. 1, the one or more fabric panels 2 will have an exterior side 9 facing away from the wearer, and an interior side 8 facing in a direction towards the wearer. FIG. 2 thus displays the undergarment 1 in a flat laid-out state as seen from the interior side.

Further, a transversal crotch axis x of the undergarment 1 is defined in a direction extending between the leg openings 6a, 6b and so as to divide the intermediate region 7 into a front intermediate region 7a extending longitudinally between the transversal crotch axis x and a front end 61a, 61b of each leg opening 6a, 6b, and a rear intermediate region 7b extending longitudinally between the transversal crotch axis x and a rear end 62a, 62b of each leg opening 6a, 6b, the transversal crotch axis x being perpendicular to the central longitudinal axis y.

As such, the transversal crotch axis x divides each leg opening 6a, 6b into a front portion of the leg opening 6a, 6b, which is intended to extend from the crotch of the user towards the front of the undergarment when the undergarment 1 is worn, and a rear portion of leg opening 6a, 6b, which is intended to extend from the crotch of the user towards the rear of the undergarment when the undergarment 1 is worn. As such, the transversal crotch axis x is to extend over a central crotch area of the user when the undergarment 1 is worn.

The absorbent undergarment 1 may comprise a waist band 14, arranged along the waist opening 5 of the absorbent undergarment 1.

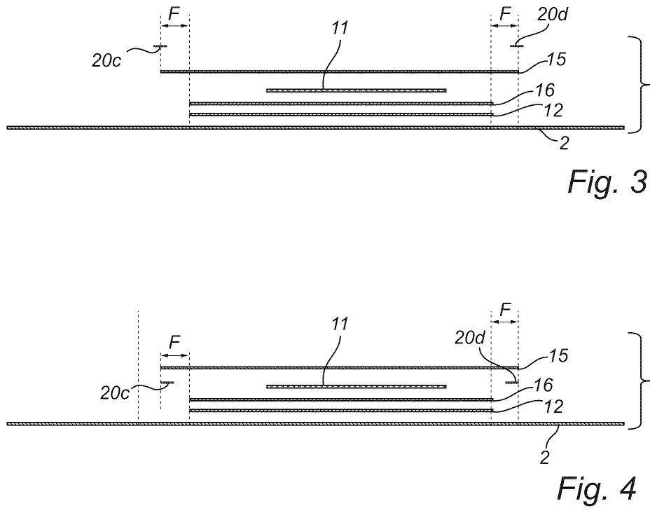

The undergarment 1 further comprises an absorbent assembly 10. As shown in FIG. 3, the absorbent assembly 10 comprises a wearer facing top layer 15, a moisture barrier 12, and two intermediate layers 11, 16 being superimposed between the top layer 15 and the moisture barrier 12 along a height axis z perpendicular to the central longitudinal axis y and the transversal crotch axis x. The layers 11, 12, 15, 16 of the absorbent assembly 10 will be further described in the below in relation to FIG. 3.

As seen in FIG. 2, in the illustrated undergarment 1, the absorbent assembly 10 is located on an interior side of the at least one fabric panel 2. The absorbent assembly 10 is arranged over the transversal crotch axis x and to extend in the front intermediate region 7a, and in the rear intermediate region 7b.

Although in some variants the absorbent assembly 10 may extend longitudinally beyond the front intermediate region 7a in a direction towards the front of the undergarment 1, in the illustrated variant, the absorbent assembly 10 extends no further than the front intermediate region 7a in the longitudinal direction towards the front region 3 of the undergarment 1.

The longitudinal extension of the absorbent assembly 10 into the rear intermediate region 7b may depend for example on the design of the undergarment 1. In some variants, such as in the illustrated variant, the absorbent assembly 10 may extend no further than the rear intermediate region 7b in the longitudinal direction towards the rear region 4 of the undergarment 1.

As such, the absorbent assembly 10 may be confined to the intermediate region 7, wherein the need for absorbance is immediately prevalent. However, for example for undergarments intended primarily for night-time use, the absorbent assembly 10 may extend beyond the rear intermediate region 7b and into the rear region 4 of the undergarment 1, and/or beyond the front intermediate region 7a and into the front region 3 of the undergarment 1.

As exemplified by the illustrated undergarment 1, the absorbent assembly 10 forms a front edge 21c directed towards the front of the undergarment 1, a rear edge 21d directed towards the rear of the undergarment, and a pair of side edges 21a, 21b. Each side edge 21a, 21b is at least partly directed towards a respective leg opening 6a, 6b. Each side edge 21a, 21b, thus connects the front edge 21c and the rear edge 21d. Each side edge 21a, 21b may be at least partly arranged along the respective leg opening 6a, 6b of the undergarment 1, such as at the leg opening 6a, 6b of the undergarment 1. In some variants, and in the illustrated example, the entire side edges 21a, 21b are arranged at the respective leg openings 6a, 6b of the undergarment 1, i.e. the side edges 21a, 21b follow the contour of the leg openings 6a, 6b of the undergarment 1.

The shape of the absorbent assembly 10 may be adapted to various absorption needs and to various designs of the undergarment 1, to provide sufficient absorption and satisfactory fit.

For example, the side edges 21a, 21b may comprise concave portions, as seen towards the central longitudinal axis y. For example, as in the illustrated variant, the entire side edges 21a, 21b are concave towards the central longitudinal axis y, i.e. curving inwards towards the central longitudinal axis y. Thus, the absorbent assembly 10 may be adapted to fit between the wearer's legs.

The front edge 21c of the absorbent assembly may similarly be shaped in view of the need for comfort and fit of the undergarment 1. In some variants, such as in the illustrated undergarment 1, the front edge may be convex from the transversal crotch axis x, i.e. curving away from the transversal crotch axis x. As illustrated in FIG. 2, at the location of the central longitudinal axis y, the front edge 21c may thus protrude towards the front of the undergarment 1 beyond the extension of the side edges 21a, b of the absorbent assembly 10. This may be beneficial for the fit of the undergarment to the body and contribute to the avoidance of unwanted creases in the fabric panels.

As mentioned in the above, each side edge 21a, 21b may at least partly be arranged at the respective leg opening 6a, 6b of the undergarment 1. This implies that the outer edge of the undergarment 1 forming each leg opening 6a, 6b at least partly comprises the side edge 21a, 21b of the absorbent assembly 10. For example, as in the illustrated variant, the outer edge of the undergarment 1 forming each leg opening 6a, 6b comprises the entire side edge 21a, 21b of the absorbent assembly 10. However, in other variants, the undergarment 1 may extend transversally beyond the absorbent assembly 10 in the directions towards each leg opening 6a, 6b in at least a part of the undergarment 1. For example, the side edges 21a, 21b of the absorbent assembly 10 may follow the leg openings 6a, 6b in a portion of the undergarment 1 adjacent the transversal crotch axis x, but the side edges 21a, 21b of the absorbent assembly 10 may deviate from the leg openings 6a, 6b in an area further to the front of the undergarment 1. In other words, the one or more fabric panels 2 may extend beyond the side edges 21a, 21b in directions away from the central longitudinal axis y. In such variants, the portions of the fabric panels 2 extending beyond the side edges 21a, 21b in directions away from the central longitudinal axis y may for example form leg portions of the undergarment 1. As such, the leg portions extending beyond the absorbent assembly 10 may adapt to and fold towards the wearer's legs, while the absorbent assembly 10 remains unfolded between the wearer's legs.

Further, when each side edge 21a, 21b is at least partly arranged at the respective leg opening 6a, 6b of the undergarment 1, as in the illustrated undergarment 1, a minimum assembly crotch width A of the absorbent assembly, i.e. a minimum distance between the side edges 21a, 21b of the absorbent assembly 10, may be equal to a minimum undergarment crotch width of the intermediate region 7 of the undergarment 1.

In some undergarment designs suitable for the absorbent undergarment proposed herein, the intermediate region 7 displays a relatively narrower crotch portion, which crotch portion comprises the transversal crotch axis x, and relatively wider front and rear portions towards the front and rear, respectively, of the crotch portion. As such, the absorbent assembly 10 may have a minimum transversal width being the transversal assembly crotch width A at the transversal crotch axis x, and the width of the absorbent assembly 10 may increase along the longitudinal axis towards the front of the undergarment, from the minimum transversal assembly crotch width A to a front portion maximum width C. Also, the width of the absorbent assembly 10 may increase along the longitudinal axis y towards the rear of the undergarment 1, from the transversal assembly crotch width A to a rear portion maximum width.

The absorbent assembly 10 is permanently attached to at least one out of the one or more fabric panels 2 in at least part of the intermediate region 7 by a set of one or more bonding members 20a, 20b, 20c, 20d.

Further, the bonding members 20a, 20b, 20c, 20d may be any type of bonding member suitable for accomplishing the permanent attachment of the absorbent assembly 10 to the one or more fabric panels 2. As such, the set of bonding members 20a, 20b, 20c, 20d may comprise one or more adhesive bonding members, such as adhesive layers, strings or dots, and/or adhesive tape such as elastic bonding tape, sealing tape, glue tape or an elastic film.

The set of bonding members 20a, 20b, 20c, 20d could be applied for example as a bonding pattern or layer formed over the surface of the absorbent assembly 10 facing the one or more fabric panels 2. As such, the set of bonding members may comprise an adhesive layer or a pattern of adhesive dots spread over the area of the surface of the absorbent assembly 10 facing the one or more fabric panels 2.

In another example, and as in the illustrated variant, the set of bonding members 20a, 20b, 20c, 20d may for example be arranged to permanently attach the absorbent assembly 10 to the at least one out of the one or more of the fabric panels 2 along at least part of one or more of the side edges 21a, 21b, the front edge 21c and the rear edge 21d.

For example, as in the illustrated variant, the set of bonding members 20a, 20b, 20c, 20d may be arranged to attach the side edges 21a, 21b, the front edge 21c and the rear edge 21d to the one or more fabric panels 2.

The bonding members 20a, 20b, 20c, 20d are arranged to extend adjacent the respective edges 21a, 21b, 21c, 21d of the absorbent assembly 10, or other variants may be envisaged where the bonding lines 20a, 20b, 20c, 20d are arranged at the respective edges 21a, 21b, 21c, 21d, e.g. so as to extend over the respective edges 21a, 21b, 21c, 21d.

The one or more bonding lines 20a, 20b, 20c, 20d may be continuous bonding lines, such as continuous adhesive lines. In other variants, however, the one or more bonding lines may be intermittent bonding lines.

The bonding members 20a, 20b, 20c, 20d may be arranged to directly attach the absorbent assembly 10 to the one or more fabric panels 2. Alternatively, the bonding members 20a, 20b, 20c, 20d may be arranged to indirectly attach the absorbent assembly 10 to the one or more fabric panels 2. For example, the bonding members 20a, 20b, 20c, 20d may be arranged to attach the absorbent assembly 10 to the one or more fabric panels 2 via an auxiliary element such as for example an edging or a reinforcement patch.

The one or more of the bonding members 20c, 20d is arranged to bond the top layer 15 (see FIG. 3) of the absorbent assembly, 10 while attaching the absorbent assembly 10, to the one or more fabric panels 2. Another bonding member may join layers 11, 16, 12 together and to seal their edges towards leakages.

FIG. 3 shows as an example of a schematic cross-sectional view along the longitudinal axis y of a portion of the intermediate region 7 of the absorbent undergarment 1 of FIGS. 1 and 2. The absorbent assembly 10 comprises a wearer facing top layer 15, a moisture barrier 12, and at least two intermediate layers 11, 16 being superimposed between the top layer 15 and the moisture barrier 12 along a height axis z perpendicular to the central longitudinal axis y and the transversal crotch axis x. In FIG. 3, the layers 11, 12, 15, 16 are illustrated as being spaced from for each other for better visibility. However, in practice the layers will be superimposed over one another, each layer contacting the neighboring upper layer and/or lower layer as seen in the height direction z. In the variant of FIGS. 3 and 4, two intermediate layers 11, 16, in this case one absorbent layer 11 and a wicking layer 16, are shown as an example. Further, the absorbent assembly 10 comprises a liquid-impermeable moisture barrier 12. The liquid-impermeable barrier 12 prevents liquid from leaking through the one or more intermediate layers 11 into the fabric panel 2. The liquid-impermeable barrier 12 may be in the form of a liquid-impermeable barrier layer 12 as illustrated in FIGS. 3 and 4. In other variants however, the liquid-impermeable barrier 12 may be a coating on the wearer facing side of a fabric panel 2 or on a garment facing side of an absorbent layer 11. The absorbent assembly 10 further comprises a top layer 15 facing the skin of the user and which is connected by bonding members 20c, 20d to the fabric panel in the extension F extending beyond the longitudinal length of the barrier 12 and any intermediate layer 11, 16 of the absorbent assembly 10. The bonding member 20c, 20d is in FIG. 3 in the form of fastening tape overlapping a border between an outer edge region of the extension F of the top layer 15 and a fabric panel 2. In FIG. 4 the fastening member 20c, 20d is a tape located between the top layer 15 and a fabric panel 2 near an edge region of the extending top layer 15.

The disclosure may be varied within the scope of the appended claims. For example, the materials and dimensions used for the different layers forming the absorbent insert may be varied, as indicated above.

1. A washable and reusable undergarment, comprising one or more fabric panels forming a front region, a back region and an intermediate region extending between the front and back regions, the front region and the back region being joined such that the undergarment forms a waist opening and a pair of leg openings,
wherein a central longitudinal axis of the undergarment is defined along the one or more fabric panels of the undergarment from the back region and towards the front region, and a transversal crotch axis of the undergarment is defined in a direction extending between the leg openings and so as to divide the intermediate region into a front intermediate region extending longitudinally between the transversal crotch axis and a front end of each leg opening, and a rear intermediate region extending longitudinally between the transversal crotch axis and a rear end of each leg opening, the transversal crotch axis being perpendicular to the central longitudinal axis;
the undergarment further comprising an absorbent assembly comprising a wearer facing top layer of a textile material, a moisture barrier, and at least one intermediate layer being superimposed along a height axis, perpendicular to the longitudinal axis and the transversal crotch axis, between the top layer and the moisture barrier, the absorbent assembly is permanently attached to at least one out of the one or more of fabric panels in at least part of the intermediate region by a set of one or more bonding members,
wherein the top layer and/or the one or more fabric panels are of natural material and the top layer has length in the longitudinal direction and an extension beyond the longitudinal length of the barrier and the intermediate layer(s) of the absorbent assembly.

2. Undergarment according to claim 1, wherein the extension of the top layer is at least 5 mm longer than the barrier and the intermediate layer(s) of the absorbent assembly.

3. Undergarment according to claim 1, wherein the extension of the top layer is at least 10 mm longer than the barrier and the intermediate layer(s) of the absorbent assembly.

4. Undergarment according to claim 1, wherein the extension of the top layer is less than 100 mm longer than the barrier and the intermediate layer(s) of the absorbent assembly.

5. Undergarment according to claim 1, wherein the extension of the top layer is less than 30 mm longer than the barrier and the intermediate layer(s) of the absorbent assembly.

6. Undergarment according to claim 1, wherein the extension of the top layer is within the front and rear intermediate regions of the undergarment.

7. Undergarment according to claim 1, wherein the top layer and the one or more fabric panels are of natural textile material.

8. Undergarment according to claim 1, wherein natural materials are selected from the group consisting of cotton, wool, silk, cellulose, regenerated cellulose, rayon, viscose, modal, lyocell, Tencel, bamboo, hemp, flax, ramie, coir or banana.

9. Undergarment according to claim 1, wherein the extension of the top layer is attached to the one or more fabric panels by the one or more bonding members selected from a fastening tape, an area of adhesive or formed at least partially via a thermocompression process.

10. Undergarment according to claim 9, wherein the fastening tape is an elastic bonding tape, sealing tape, glue tape or an elastic film.

11. Undergarment according to claim 9, wherein the bonding member is overlapping a border between an outer edge region of the extension of the top layer and a fabric panel.

12. Undergarment according to claim 9, wherein the bonding member is located between the top layer and a fabric panel near an edge region of the extending top layer.

13. Undergarment according to claim 9, wherein the thermocompression process is by applying heat and/or pressure to adhesive material during the manufacture of the undergarment.

14. Undergarment according to claim 1, wherein the at least one intermediate layer is an absorbent layer.

15. Undergarment according to claim 1, wherein the undergarment can be washed, at least at 30° C., and be reused at least 10 times.

16. Undergarment according to claim 1, wherein the extension of the top layer is attached to the one or more fabric panels by the one or more bonding members.
